(19) United States
(12) Patent Application Publication
(10) Pub. No.: US 2025/0331792 A1
(43) Pub. Date: Oct. 30, 2025

(54) COMBINATION OF X-RAY INSTALLATIONS TO FORM AN X-RAY SYSTEM

(71) Applicant: Siemens Healthineers AG, Forchheim (DE)

(72) Inventors: Jan Paul GUERA, Oberursel (DE); Claudia IGNEY, Erlangen (DE); Patrik SCHWINGENSCHLOEGL, Fuerth (DE); Joerg HOFMANN, Forchheim (DE)

(73) Assignee: Siemens Healthineers AG, Forchheim (DE)

(21) Appl. No.: 19/186,801

(22) Filed: Apr. 23, 2025

(30) Foreign Application Priority Data
Apr. 25, 2024 (DE) 10 2024 203 862.8

(51) Int. Cl. A61B 6/00; A61B 6/10

(52) U.S. Cl. CPC A61B 6/4441; A61B 6/105; A61B 6/4405

(57) ABSTRACT

An X-ray installation has a base unit on which a C-arm is arranged. An X-ray source and an X-ray detector are arranged on the C-arm. The C-arm forms half of a closed, convex, point-symmetrical curve. The X-ray source and/or the X-ray detector is/are displaceable along the C-arm. In this way, an angle formed by the X-ray source and the X-ray detector with an isocenter of the full circle is adjustable to a minimum value that is less than 180°. Two such X-ray installations can form an X-ray system in which the two X-ray installations are arranged and adjusted such that the closed, convex, point-symmetrical curves of the two X-ray installations form an integrated, closed, point-symmetrical, convex curve and the C-arms of the two X-ray installations form complementary halves of the integrated, closed, convex, point-symmetrical curve.

### CROSS-REFERENCE TO RELATED APPLICATION(S)

The present application claims priority under 35 U.S.C. § 119 to German Patent Application No. 10 2024 203 862.8, filed Apr. 25, 2024, the entire contents of which are incorporated herein by reference.

### FIELD

Independent of the grammatical term usage, individuals with male, female or other gender identities are included within the term.

One or more example embodiments relates to an X-ray installation, wherein the X-ray installation has a base unit on which a C-arm is arranged, wherein an X-ray source and an X-ray detector are arranged on the C-arm.

One or more example embodiments furthermore relates to an X-ray system comprising two X-ray installations.

### RELATED ART

X-ray installations are known.

DE 10 2010 020 605 A1 and the corresponding U.S. Pat. No. 8,662,750 B2 disclose a CT system that has a substantially ring-shaped gantry. The gantry has a fold-out segment such that the ring-shaped gantry can be opened and closed. In the open state, a patient can be introduced into and removed from the space enclosed by the ring-shaped gantry. In the closed state, the usual examinations are carried out. Such an X-ray installation is also distributed under the name O-arm by Medtronic plc, Dublin, Ireland.

DE 10 2012 211 330 A1 and the corresponding U.S. Pat. No. 9,055,912 B2 disclose an X-ray installation that has a C-arm arranged on a base unit, on which arm are arranged an X-ray source and an X-ray detector. The C-arm is divided into an upper and a lower segment that can be pivoted open and pivoted shut again at one end. In the pivoted-shut state, the C-arm forms a full circle on which the X-ray source and X-ray detector can be moved.

Brainlab AG, Olof-Palme-Straße 9, 81829 Munich, Germany distributes an X-ray installation under the name Loop-X that has a base unit on which a closed circular ring is arranged. An X-ray source and an X-ray detector are arranged on the ring, wherein these two components can be displaced mutually independently on the ring.

Brainlab AG furthermore distributes a CT system under the name Airo that has a base unit on which a closed circular gantry is arranged. An X-ray source and an X-ray detector are arranged on the gantry, wherein these two components can revolve about an isocenter of the gantry, such that, with regard to the isocenter, they are always located opposite one another.

EP 3 135 200 A1 discloses an X-ray installation that has a base unit on which a C-arm is arranged. The C-arm forms somewhat more than half a full circle. An X-ray source and an X-ray detector are arranged on the C-arm, wherein the X-ray source and X-ray detector form an angle of 180° with an isocenter of the full circle. A further C-arm is mounted on the C-arm such that the further C-arm constitutes an adjustable extension of the first-stated C-arm. In this way, the angular range over which the entirety of the two C-arms extend around the isocenter can, if required, be enlarged to just about 360°. A further X-ray detector is arranged at the end of the further C-arm. A further X-ray source is also arranged either fixedly at the other end of the further C-arm or movably on the first-stated C-arm, such that the further X-ray source and the further X-ray detector form an angle of 180° with the isocenter of the full circle.

EP 3 598 947 A1 and the corresponding U.S. Pat. No. 10,595,799 B2 disclose an X-ray installation of the above-stated type. These two documents also disclose an X-ray system that consists of two such X-ray installations. The two X-ray installations can be operated independently of one another or together. If they are operated together, the control device of one of the two X-ray installations acts as the master for the two X-ray installations and the control device of the other X-ray installation acts as a slave. The two C-arms may, for example, be arranged and adjusted such that, for one of the X-ray installations, the X-ray source is above and the X-ray detector below an object under examination, such that the beam path for this X-ray installation is substantially vertical, and, for the other X-ray installation, the X-ray source and the X-ray detector are arranged on both sides of the object under examination, such that the beam path for this X-ray installation is substantially horizontal.

Most X-ray installations and X-ray systems, and this applies equally to permanently installed installations and systems and to mobile installations and systems, are limited in terms of their imaging options. They are also often complex to install and operate. For example, some X-ray installations are suitable only for 2D imaging or only for 3D imaging or only for biplanar transillumination with a fixed 90° offset geometry. Dual-energy imaging is also often not possible.

Some of the above-stated problems can be solved by using higher grade systems. Examples of such systems are intraoperative MR/CT systems and other OP installations. However, these systems are complex and costly to use. These system are also not mobile but always located in the same room.

The X-ray installations and X-ray systems known from EP 3 598 947 A1 offer some progress in this respect. Two X-ray installations can be combined to form an integrated X-ray system. Dual-energy imaging is also possible.

### SUMMARY

In the X-ray system known from EP 3 598 947 A1, the two sub-installations can be used not only together in coordinated manner but also independently and separately from one another. However, correctly arranging the two X-ray installations relative to one another so that they can be used together in coordinated manner is complex, troublesome, and error-prone.

One or more example embodiments provides possible ways via which the two X-ray installations can correctly be arranged relative to one another in a straightforward manner.

This may be achieved by an X-ray installation having the features of claim 1. Advantageous embodiments of the X-ray installation are the subject matter of dependent claims 2 and 3.

### BRIEF DESCRIPTION OF THE DRAWINGS

Characteristics, features and advantages and the manner in which these are achieved will become clearer and more distinctly comprehensible from the following description of the exemplary embodiments, which are explained in greater detail in connection with the drawings, in which in schematic representation:

FIG. 1 shows an X-ray installation according to one or more example embodiments,

FIGS. 2 to 5 show a C-arm of the X-ray installation of FIG. 1 according to one or more example embodiments,

FIG. 6 shows a further X-ray installation according to one or more example embodiments,

FIG. 7 shows an X-ray system with two X-ray installations according to one or more example embodiments,

FIG. 8 shows a control system for the X-ray installations of FIG. 7 according to one or more example embodiments,

FIG. 9 shows a modification of the X-ray system of FIG. 7 according to one or more example embodiments,

FIGS. 10 to 13 show various states of the X-ray system of FIG. 7 according to one or more example embodiments,

FIG. 14 shows a side view of the upper ends of two C-arms according to one or more example embodiments,

FIG. 15 shows a top view of the upper ends of the two C-arms of FIG. 14 according to one or more example embodiments, and

FIG. 16 shows a further X-ray system with two X-ray installations according to one or more example embodiments.

### DETAILED DESCRIPTION

According to one or more example embodiments, an X-ray installation of the above-stated type is configured in that the C-arm forms half of a closed, convex, point-symmetrical curve and in that the X-ray source and/or the X-ray detector is/are displaceable along the C-arm, such that an angle formed by the X-ray source and the X-ray detector with an isocenter of the closed, convex, point-symmetrical curve is adjustable to a minimum value that is less than 180°. It is particularly preferred for both the X-ray source and the X-ray detector to be displaceable on the C-arm.

The closed, convex, point-symmetrical curve can be configured as required. For example, it may be a circle or an ellipse or an oval. The C-arm may optionally have a straight end portion at one or both ends. The end portion may optionally also be travelable/extendable or insertable/removable.

When the angle is adjusted to 180°, conventional operation with isocentric imaging is possible using the X-ray installation. When the angle is adjusted to a value of below 180°, operation with non-isocentric imaging is possible using the X-ray installation.

Because the C-arm extends over half the closed, convex, point-symmetrical curve, it is possible to arrange two such X-ray installations such that the two C-arms form the two halves of a common, integrated, closed, convex, point-symmetrical curve. This embodiment facilitates correct arrangement of the two X-ray installations relative to one another. Thanks to the possibility of displacing the X-ray sources and/or the X-ray detectors of the two X-ray installations on the respective C-arm, it is possible to arrange the X-ray sources and the X-ray detectors in pairs such that the X-ray sources of the two X-ray installations irradiate the X-ray detector of the respective other X-ray installation.

What is important here is to be able to displace the X-ray source and/or the X-ray detector of the respective X-ray installation along the C-arm. It must therefore be possible not to move the C-arm itself but only to displace the X-ray source and/or the X-ray detector. It may, of course, also be possible, as also in the prior art, to displace the entire C-arm about the isocenter. However, this displaceability, if implemented, is not an alternative but an addition to the displaceability of the X-ray source and/or the X-ray detector along the C-arm.

The minimum value can be set as required. The minimum value is preferably 150° or below, preferably 120° or below, and in particular 100° or below. It is even possible for the minimum value to be 90° or below. As a result, when two such X-ray installations are combined to form an X-ray system, in particular biplanar image acquisition with orthogonal imaging geometries becomes possible.

The X-ray installation is preferably configured as a mobile X-ray installation. As a result, the X-ray installation can be put to particularly versatile use.

Even when such an X-ray installation is used in stand-alone mode, it has the advantage over a conventional X-ray installation that non-isocentric imaging is possible in addition to isocentric imaging. The X-ray installation according to one or more example embodiments demonstrates its full advantages, however, when it is combined with a further X-ray installation according to one or more example embodiments to form a corresponding X-ray system. In this case, the two X-ray installations are arranged and adjusted such that the full circle of the two X-ray installations forms an integrated, full circle and the C-arms of the two X-ray installations form complementary halves of the integrated, full circle. Furthermore, control devices of the two X-ray installations are coupled to one another such that the two X-ray installations are operated in coordinated manner. Finally, the X-ray sources of the two X-ray installations irradiate the X-ray detector of the respective other X-ray installation. Such irradiation, it goes without saying, is only possible if an angle formed by the X-ray source and X-ray detector of the respective X-ray installation with the isocenter of the integrated, full circle is less than 180°. It is for this reason that the displaceability of the respective X-ray source and/or of the respective X-ray detector on the respective C-arm is necessary, such that the angle can be adjusted between 180° and the minimum value.

The X-ray sources and/or the X-ray detectors of the C-arm of the respective X-ray installation can change over to the C-arm of the respective other X-ray installation. This enables highly flexible operation of the X-ray system.

This operation is particularly flexible when the X-ray sources and the X-ray detectors of both X-ray installations are positionable at any desired position on the integrated, full circle. In this way, similarly to a CT system, it is for example possible to cause the X-ray sources and the X-ray detectors to revolve about the isocenter of the integrated, full circle, optionally even over a number of complete revolutions. The X-ray sources and X-ray detectors can be supplied with the necessary power for example via circumferential sliprings. Data transmission may be contactless or with-contact via circumferential data transmission elements. The X-ray sources and X-ray detectors can be purely mechanically guided via rail systems that are arranged in the C-arms and merge into one another.

The C-arms preferably have positioning aids, by way of which the C-arm of the one X-ray installation is oriented relative to the C-arm of the other X-ray installation when the C-arms of the two X-ray installations are being brought together. Corresponding devices are known to persons skilled in the art. For example, the devices may be configured as fixed or retractable or extendable centering pins on the C-arm of the one X-ray installation that are introduced into corresponding recesses on the C-arm of the other X-ray installation. Alternatively, the devices may for example be configured as electromagnetically interacting parts. Other embodiments are also possible.

The C-arms of the two X-ray installations preferably have releasable locking means, by way of which the C-arm of the one X-ray installation is fixable relative to the C-arm of the other X-ray installation. These ensure that, once the (correct) orientation of the C-arms of the X-ray installations relative to one another is established, it is not accidentally overridden again.

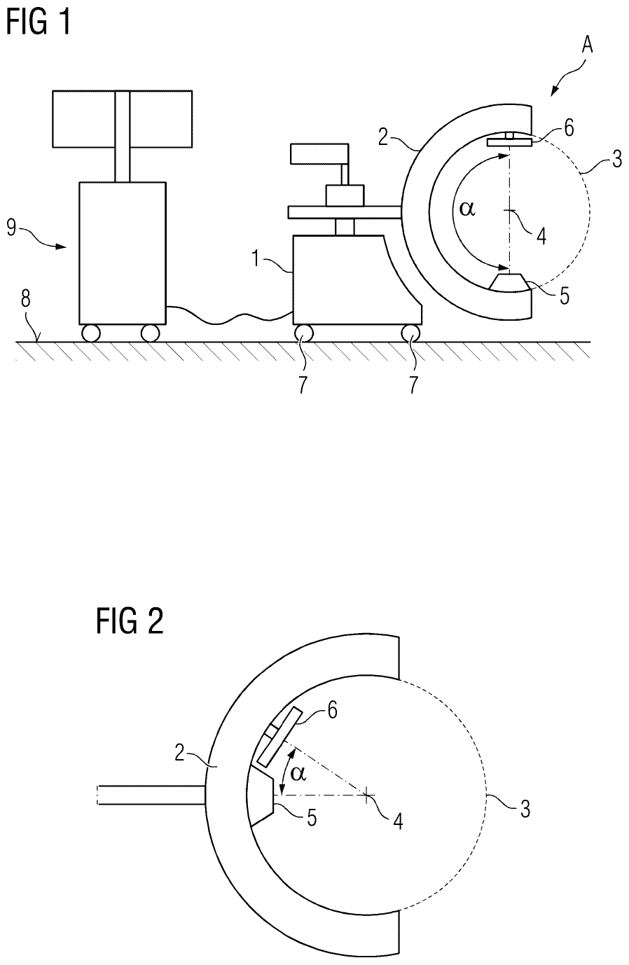

According to FIG. 1, an X-ray installation A has a base unit 1. A C-arm 2 is arranged on the base unit 1. The C-arm 2 forms half of a full circle or generally a closed, convex, point-symmetrical curve 3. In the case of a full circle with at least substantially constant spacing, it thus extends over 180° about an isocenter 4 of the closed, convex, point-symmetrical curve 3. An X-ray source 5 and an X-ray detector 6 are arranged on the C-arm 2. As depicted in FIG. 1, the X-ray source 5 and X-ray detector 6 are arranged at the ends of the C-arm 2, such that an angle α formed by the X-ray source 5 and the X-ray detector 6 with the isocenter 4 is at its maximum value of 180°.

The X-ray source 5 and/or the X-ray detector 6, preferably both the X-ray source 5 and the X-ray detector 6, are displaceable along the C-arm 2. As a result, the angle α can also have a value of less than 180°. FIG. 2 shows an embodiment in which both the X-ray source 5 and the X-ray detector 6 have been displaced along C-arm 2 and the angle α is adjusted to its minimum value. According to FIG. 2, the minimum value is below 90°. However, the minimum value may also in principle have a different value. The minimum value is preferably 150° or below, for example 120° or below, and in particular 100° or below. If possible, effort should be made to ensure that a minimum value of at least 90° can be achieved. As depicted in FIG. 2, the minimum value may be so small that the X-ray source 5 and X-ray detector 6 immediately adjoin one another.

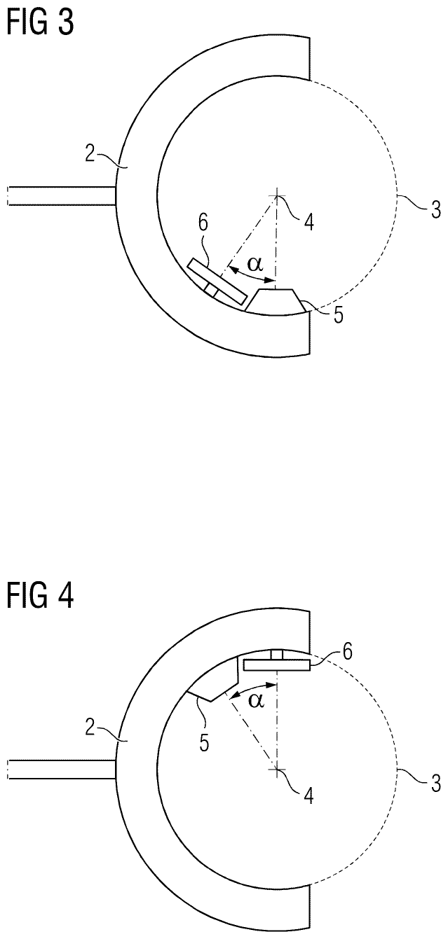

FIGS. 3 and 4 likewise show a situation in which the angle α is adjusted to its minimum value. In FIG. 3, however, the X-ray source 5 is located at its end of the C-arm 2 and the X-ray detector 6 is moved as far as possible toward the X-ray source 5. The opposite is the case in FIG. 4.

In addition to the displaceability of the X-ray source 5 and/or X-ray detector 6 along the C-arm 2, the entire C-arm 2 can, see FIG. 5, be moved relative to the base unit 1. This movement is, however, a movement that differs from the displacement of the X-ray source 5 and/or X-ray detector 6 along the C-arm 2. Various degrees of freedom can be provided for the displacement of the C-arm 2, for example displacement along the full circle 3 or rotation of the C-arm 2 or a lengthwise or transverse shift of the C-arm 2. These displacement options are generally known to persons skilled in the art. The depiction in FIG. 5, in which the entire C-arm 2 is displaced by 45° along the closed, convex, point-symmetrical curve 3, is purely exemplary.

The entire X-ray installation A of FIGS. 1 to 5 is preferably mobile. The X-ray installation A can, for example, have wheels 7, by way of which the X-ray installation A is displaceable on a floor 8. In many cases, the X-ray installation A furthermore has an assigned monitor station 9. The monitor station 9 can be integrated in the base unit 1 or be a separate unit.

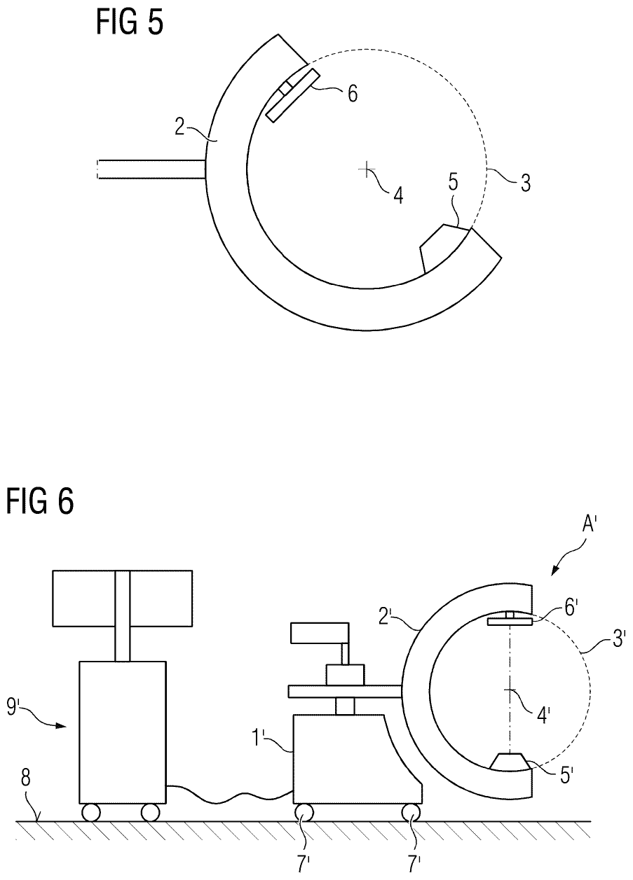

FIG. 6 shows a further X-ray installation, A′. The X-ray installation A′ of FIG. 6 may be configured identically to the X-ray installation A of FIGS. 1 to 5. The components denoted 1′ to 7′ and 9′ in FIG. 6 correspond 1:1 with components 1 to 7 and 9 of X-ray installation A of FIGS. 1 to 5. Same applies to the angle α′. The floor 8 is, of course, the same as in FIGS. 1 to 5.

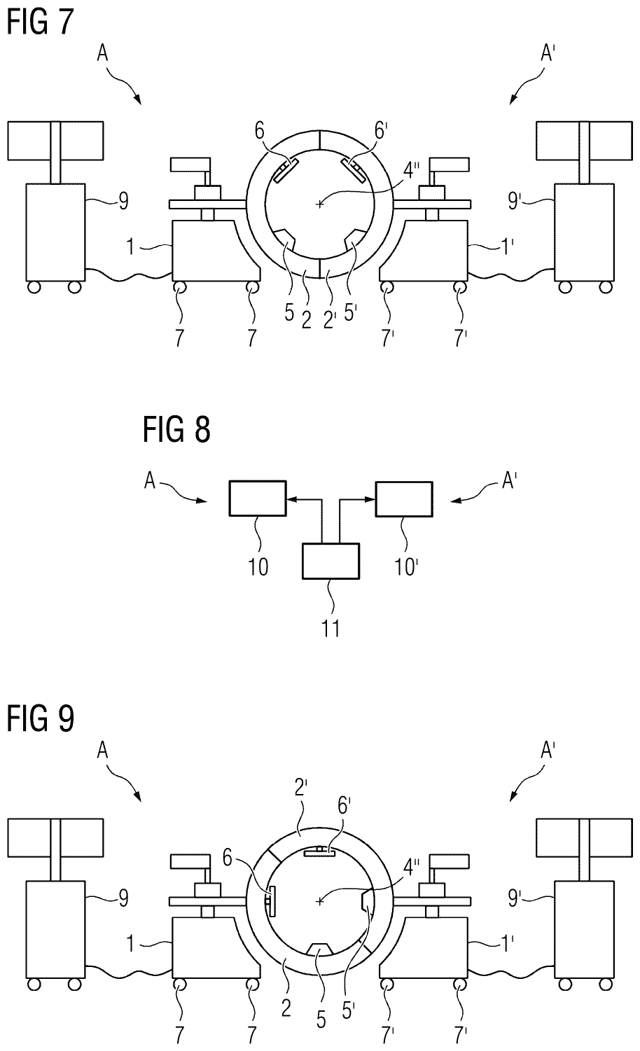

FIG. 7 shows an X-ray system, both the X-ray installation A of FIGS. 1 to 5 and the X-ray installation A′ of FIG. 6. According to FIG. 7, the two X-ray installations A, A′ are arranged and adjusted such that the closed, convex, point-symmetrical curves 3, 3′ of the two X-ray installations A, A′ form an integrated, closed, convex, point-symmetrical curve. The isocenter of the integrated, closed, convex, point-symmetrical curve is denoted 4″. According to FIG. 7, the C-arms 2, 2′ of the two X-ray installations A, A′ form complementary halves of the integrated, closed, convex, point-symmetrical curve. The X-ray sources 5, 5′ and the two X-ray detectors 6, 6′ are furthermore arranged such that the X-ray sources 5, 5′ of the two X-ray installations A, A′ irradiate the X-ray detector 6′, 6 of the respective other X-ray installation A′, A′. The X-ray source 5 of the one X-ray installation A thus irradiates the X-ray detector 6′ of the other X-ray installation A′ and vice versa.

According to FIG. 8, the two X-ray installations A, A′ have control devices 10, 10′. The control devices 10, 10′ are coupled to one another such that the two X-ray installations A, A′ are operated in coordinated manner. For example, the control device 10 of the one X-ray installation A can act as the master for the two X-ray installations A, A′ and the control device 10′ of the other X-ray installation A′ can act as a slave that the other X-ray installation A′ controls in accordance with the requirements of control device 10. It is likewise possible for a higher-level device 11 to be present that acts as the master for the two control devices 10, 10′, such that the two control devices 10, 10′ are slaves of the higher-level device 11. EP 3 598 947 A1 explains corresponding procedures. The higher-level device 11 may, for example, comprise a user interface.

In the simplest embodiment, the X-ray sources 5, 5′ and/or the X-ray detectors 6, 6′ are displaceable only along their respective C-arm 2, 2′. In this case, the X-ray sources 5, 5′ and the X-ray detectors 6, 6′ can, for example, be adjusted such that, with regard to the isocenter 4″, the X-ray detector 6′ is located diametrically opposite X-ray source 5 and, with regard to the isocenter 4″, X-ray detector 6 is likewise also located diametrically opposite the X-ray source 5′. Furthermore, as likewise depicted in FIG. 7, when viewed in the circumferential direction about the isocenter 4″ of the integrated, closed, convex, point-symmetrical curve, there may in each case be an angular distance between directly adjacent X-ray sources 5, 5′ and X-ray detectors 6, 6′ of exactly or at least approximately 90°.

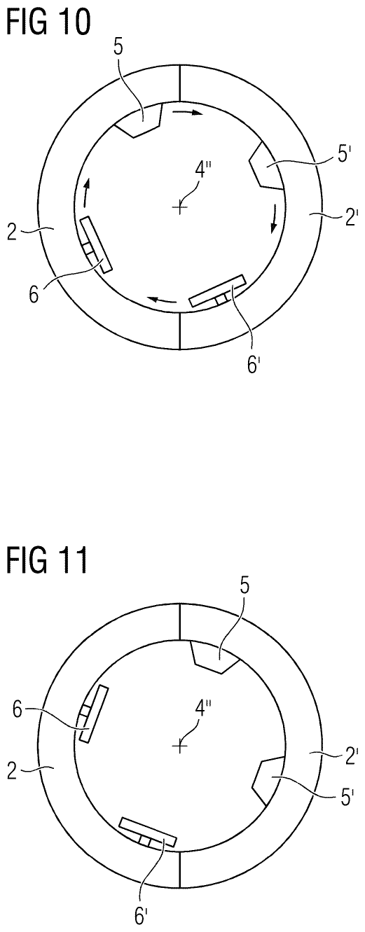

In the configuration of FIG. 7, the beam paths of the respective X-ray source 5, 5′ to the respective X-ray detector 6′, 6 are in each case oblique, i.e., neither horizontal nor vertical. This can, however, also be achieved even in the simplest embodiment. All that is required for this purpose is, as depicted in FIG. 9, to appropriately displace both the whole C-arms 2, 2′. For example, the two C-arms 2, 2′ can be displaced by approx. 45° about the isocenter 4″ of the integrated, closed, convex, point-symmetrical curve. However, it is better if, as depicted in FIGS. 10 to 13, the X-ray sources 5, 5′ and/or the X-ray detectors 6, 6′ can change over from C-arm 2, 2′ of the respective X-ray installation A, A′ to C-arm 2′, 2 of the respective other X-ray installation A′, A. The X-ray sources 5, 5′ and X-ray detectors 6, 6′ of the two X-ray installations A, A′ are preferably even positionable at any desired position on the integrated, closed, convex, point-symmetrical curve. The necessary power supply and necessary data communication are readily implementable as such.

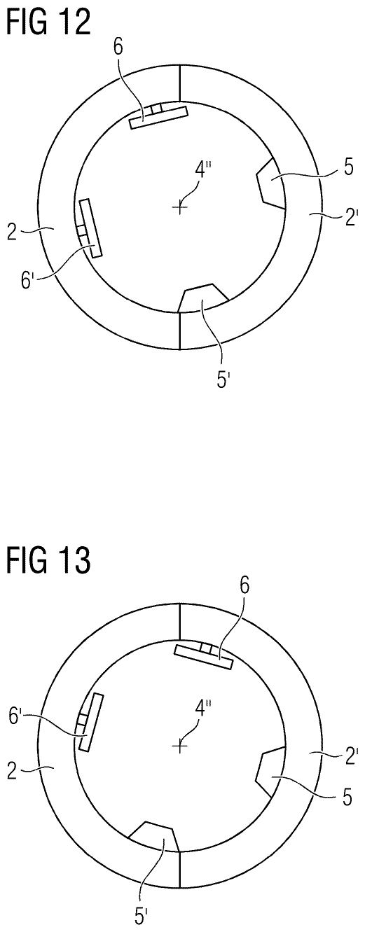

In the exemplary depiction of FIGS. 10 to 13, which depicts only C-arms 2, 2′ and X-ray sources 5, 5′ and X-ray detectors 6, 6′, the X-ray sources 5, 5′ and X-ray detectors 6, 6′ are moving uniformly and at an angular distance of 90° clockwise along the C-arms 2, 2′. In the state according to FIG. 10, X-ray source 5 is located at the upper end of C-arm 2 and X-ray detector 6′ is located at the lower end of C-arm 2′. Due to the clockwise movement, X-ray source 5 is located shortly thereafter, as depicted in FIG. 11, at the upper end of C-arm 2′ and X-ray detector 6′ is located at the lower end of C-arm 2. Similarly, according to FIG. 12, X-ray detector 6 is located at a later point in time at the upper end of C-arm 2 and X-ray source 5′ is located at the lower end of C-arm 2′. Due to the clockwise movement, X-ray detector 6 is located shortly thereafter, as depicted in FIG. 13, at the upper end of C-arm 2′ and X-ray source 5′ is located at the lower end of C-arm 2.

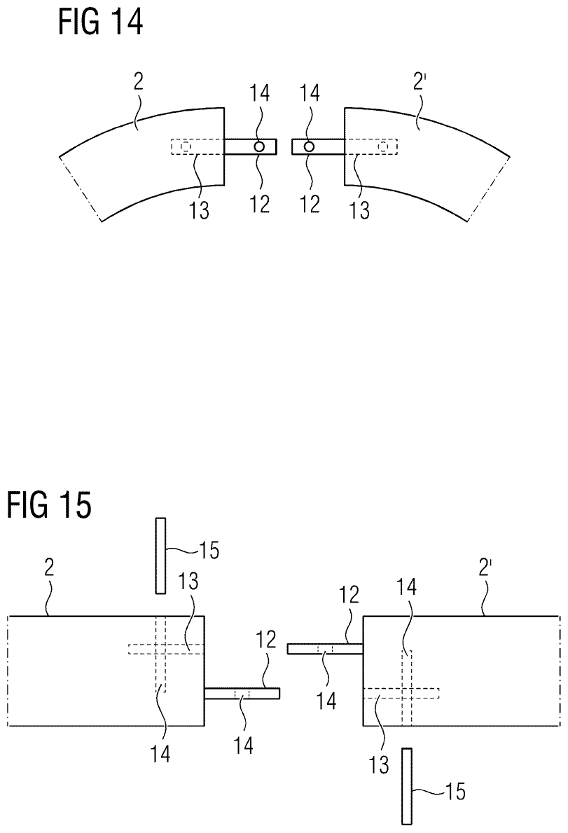

The C-arms 2, 2′ preferably have positioning aids, by way of which the C-arm 2 of the one X-ray installation A is oriented relative to the C-arm 2′ of the other X-ray installation A′ when the C-arms 2, 2′ of the two X-ray installations A, A′ are being brought together. For example, as depicted in FIGS. 14 and 15, the C-arms 2, 2′ may have cylindrical rods 12 as positioning aids that can extend into corresponding recesses 13 of the respective other C-arm 2′, 2. The rods 12 can be tapered at their front ends and/or the recesses 13 can be flared at their openings in order to facilitate introduction of the rods 12 into the recesses 13.

The C-arms 2, 2′ of the two X-ray installations A, A′ furthermore preferably have releasable locking means, by way of which C-arm 2 of the one X-ray installation A is fixable relative to C-arm 2′ of the other X-ray installation A′. For example, transverse bores 14 can be introduced into the rods 12 and into the C-arms 2, 2′, into which bores fixing pins 15 can be introduced.

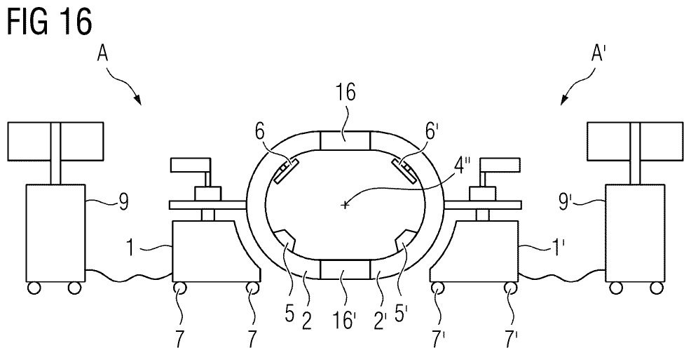

FIG. 16 shows a further X-ray system with two X-ray installations A, A′. The difference relative to the X-ray system of FIG. 7 is substantially that, in the X-ray system of FIG. 16, the C-arms 2, 2′ of the two X-ray installations A, A′ are connected together not directly but via end portions 16, 16′. In the embodiment of FIG. 16, the end portions 16, 16′ are insertable or removable. This is in particular advantageous if it is to be possible for the X-ray sources 5, 5′ and/or the X-ray detectors 6, 6′ to change over from C-arm 2, 2′ of the respective X-ray installation A, A′ to C-arm 2′, 2 of the respective other X-ray installation A′, A. This embodiment is (of course) also implementable if X-ray sources 5, 5′ and/or X-ray detectors 6, 6′ are to be displaceable only along their respective C-arm 2, 2′. However, if X-ray sources 5, 5′ and/or X-ray detectors 6, 6′ are to be displaceable only along their respective C-arm 2, 2′, it is likewise also possible for end portions 16, 16′ to be a solid, retractable, or extendable component of the respective C-arm 2, 2′.

Example embodiments have many advantages. For example, it is straightforwardly and reliably possible to combine two identically constructed X-ray installations A, A′ to form an X-ray system operable as a unit.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. s used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e. g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, operations, steps, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SOC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. An X-ray installation, comprising:
a base unit;
a C-arm on the base unit; and
an X-ray source and an X-ray detector being on the C-arm, wherein
the C-arm forms half of a closed, convex, point-symmetrical curve and at least one of the X-ray source or the X-ray detector is displaceable along the C-arm, such that an angle formed by the X-ray source and the X-ray detector with an isocenter of the closed, convex, point-symmetrical curve is adjustable to a minimum value that is less than 180°.

2. The X-ray installation of claim 1, wherein the minimum value is 150° or below.

3. The X-ray installation of claim 1, wherein the X-ray installation is a mobile X-ray installation.

4. An X-ray system comprising:
two X-ray installations, each of the two X-ray installations being the X-ray installation of claim 1, wherein
the two X-ray installations are arranged such that the closed, convex, point-symmetrical curves of the two X-ray installations form an integrated, closed, convex, point-symmetrical curve and the C-arms of the two X-ray installations form complementary halves of the integrated, closed, convex, point-symmetrical curve,
control devices of the two X-ray installations are coupled to one another such that the two X-ray installations are operated in coordinated manner, and
the X-ray sources of the two X-ray installations irradiate the X-ray detector of a respective other X-ray installation.

5. The X-ray system of claim 4, wherein at least one of the X-ray sources or the X-ray detectors of the C-arm of a respective X-ray installation can change over to the C-arm of the other X-ray installation.

6. The X-ray system of claim 5, wherein the X-ray sources and X-ray detectors of the two X-ray installations are positionable at any desired position on the integrated, closed, convex, point-symmetrical curve.

7. The X-ray system of claim 4, wherein the C-arms have positioning aids configured to orient the C-arm of one X-ray installation relative to the C-arm of the other X-ray installation when the C-arms of the two X-ray installations are being brought together.

8. The X-ray system of claim 4, wherein the C-arms of the two X-ray installations include releasable locking means configured to fix the C-arm of one X-ray installation relative to the C-arm of the other X-ray installation.

9. The X-ray installation as claimed in claim 2, wherein the minimum value is 120° or below.

10. The X-ray installation as claimed in claim 9, wherein the minimum value is 100° or below.

11. The X-ray system of claim 5, wherein the C-arms have positioning aids configured to orient the C-arm of one X-ray installation relative to the C-arm of the other X-ray installation when the C-arms of the two X-ray installations are being brought together.

12. The X-ray system of claim 6, wherein the C-arms have positioning aids configured to orient the C-arm of one X-ray installation relative to the C-arm of the other X-ray installation when the C-arms of the two X-ray installations are being brought together.

13. The X-ray system of claim 5, wherein the C-arms of the two X-ray installations include releasable locking means configured to fix the C-arm of one X-ray installation relative to the C-arm of the other X-ray installation.

14. The X-ray system of claim 6, wherein the C-arms of the two X-ray installations include releasable locking means configured to fix the C-arm of one X-ray installation relative to the C-arm of the other X-ray installation.

15. The X-ray system of claim 7, wherein the C-arms of the two X-ray installations include releasable locking means configured to fix the C-arm of one X-ray installation relative to the C-arm of the other X-ray installation.
